(19) United States
(12) Patent Application Publication
(10) Pub. No.: US 2007/0289591 A1
(43) Pub. Date: Dec. 20, 2007

(54) Endotracheal Tube Exchanger

(76) Inventor: Hortencia Clayton, Orando, FL (US)

Correspondence Address: ENRIQUE G. ESTEVEZ, 255 SOUTH ORANGE AVE, SUITE 1401, ORLANDO, FL, 32802-3791, US

(21) Appl. No.: 11/424,097

(22) Filed: Jun. 14, 2006

(51) Int. Cl. A61M 16/00; A62B 9/06

(52) U.S. Cl. 128/200.26; 128/207.14

(57) ABSTRACT

An endotracheal tube exchanger (ETX) includes a tube slide portion having an elongated lengthwise dimension having a first end and a second end, a relatively narrow width dimension having a curved cross section, and a generally arcuate shape having inner and outer peripheries wherein the curved cross section forms a concavity along the inner periphery, and a handle portion extending outwardly from the outer periphery of the first end of said tube slide portion. Associated methods describe the use of the endotracheal tube exchanger which does not require insertion of the exchanger device within the endotracheal tube to be changed or within the new replacement endotracheal tube.

### FIELD OF THE INVENTION

The present invention relates to the field of medical treatment and, more particularly, to a device which aids in exchanging an endotracheal tube in a previously intubated patient.

### BACKGROUND OF THE INVENTION

In medical treatment it is often necessary to provide the patient with forced ventilation. This may be accomplished by inserting a breathing tube into the patient's respiratory airway, also known as intubation. Such intubation may be established by the nasopharyngeal route or, more typically, by the oropharyngeal route via an endotracheal tube (ETT). Once the ETT is in place in the patient, the tube is connected to a source of oxygen enriched air under sufficient pressure to provide assisted ventilation of the patient's lungs.

At times it may be desirable or necessary to exchange the patient's existing ETT for a new one. This situation may come about because the originally placed ETT may be too small for the patient, for example, an ETT placed during emergency treatment at the scene of an accident. The ETT may develop a leaky cuff, thereby making it difficult to remain in proper placement in the patient. Additionally, the ETT may also have become occluded. ETT exchange is performed with the existing ETT has been determined to be either damaged or ineffective.

Accordingly, ETT exchange is a common procedure, particularly in the critical care areas of a hospital. In an exchange, typically the ETT is first disconnected from the source of ventilation, a guide wire, stylet or endoscope is inserted into the ETT is advanced to the end of the ETT and the ETT is then withdrawn over the insert. A new ETT is then positioned over the insert and is advanced into the patient's airway. Those skilled in the art will appreciate that this procedure, while not uncommon, is time consuming and tricky to perform.

### SUMMARY OF THE INVENTION

With the foregoing in mind, the present invention advantageously provides an endotracheal tube exchanger (ETX) which does not require insertion into the ETT as with the existing typical exchanger devices. The present ETX, therefore, allows easier and quicker ETT exchanges.

An embodiment of the invention comprises an endotracheal tube exchanger (ETX) which includes a tube slide portion having an elongated lengthwise dimension having a first end and a second end, a relatively narrow width dimension having a curved cross section, and a generally arcuate shape having inner and outer peripheries wherein the curved cross section forms a concavity along the inner periphery. The ETX also includes a handle portion extending outwardly from the outer periphery of the first end of the tube slide portion.

Another embodiment of the invention has an arcuate slide having first and second ends defining therebetween a curve having an inner periphery and an outer periphery, the inner periphery of the arcuate slide being generally concave. A handle extends away from the arcuate slide at the first end, the handle providing a sufficient extent to receive thereon at least part of an operator's hand.

The present invention also includes methods of exchanging ETTs by use of the described embodiments of the ETX.

### BRIEF DESCRIPTION OF THE DRAWINGS

Some of the features, advantages, and benefits of the present invention having been stated, others will become apparent as the description proceeds when taken in conjunction with the accompanying drawings, presented for solely for exemplary purposes and not with intent to limit the invention thereto, and in which:

FIG. 1 shows cross sectional side elevation of an intubated patient having an ETX inserted in preparation for withdrawal of the ETT according to an embodiment of the present invention;

FIG. 2 provides a perspective view of the ETX of the present invention;

FIG. 3 is a cross section of the ETX along line 3-3 as shown in FIG. 2;

FIG. 4 is a side elevation view of the ETX of the present invention, showing the relative flexibility of the arcuate tube slide portion of the device; and

FIG. 5 illustrates the ETX device of the present invention in use to extubate a patient.

### DETAILED DESCRIPTION OF PREFERRED EMBODIMENTS

The present invention will now be described more fully hereinafter with reference to the accompanying drawings, in which preferred embodiments of the invention are shown. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. Moreover, the materials, methods and examples given are illustrative in nature only and not intended to be limiting. Accordingly, this invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art. Other features and advantages of the invention will be apparent from the following detailed description, and from the claims.

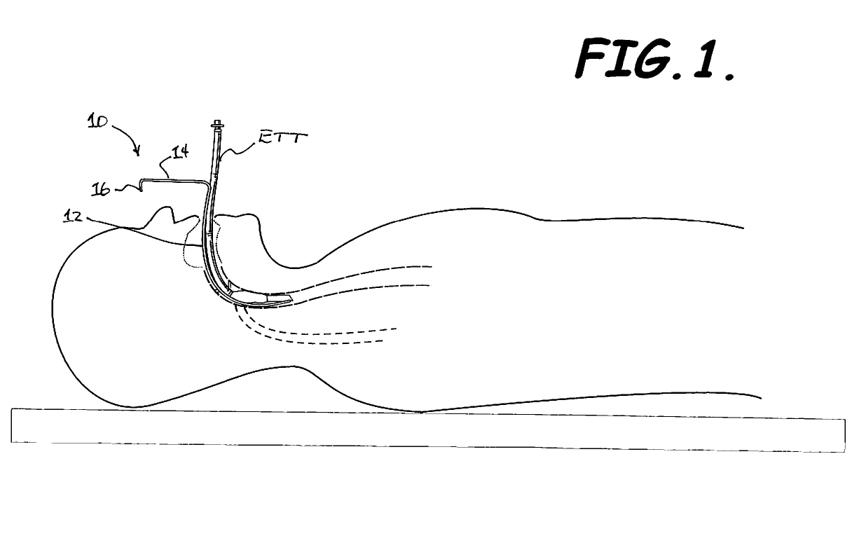

FIGS. 1-5 illustrate various structural and functional aspects of the present invention, which comprises an endotracheal tube exchanger (ETX) and its associated methods of use.

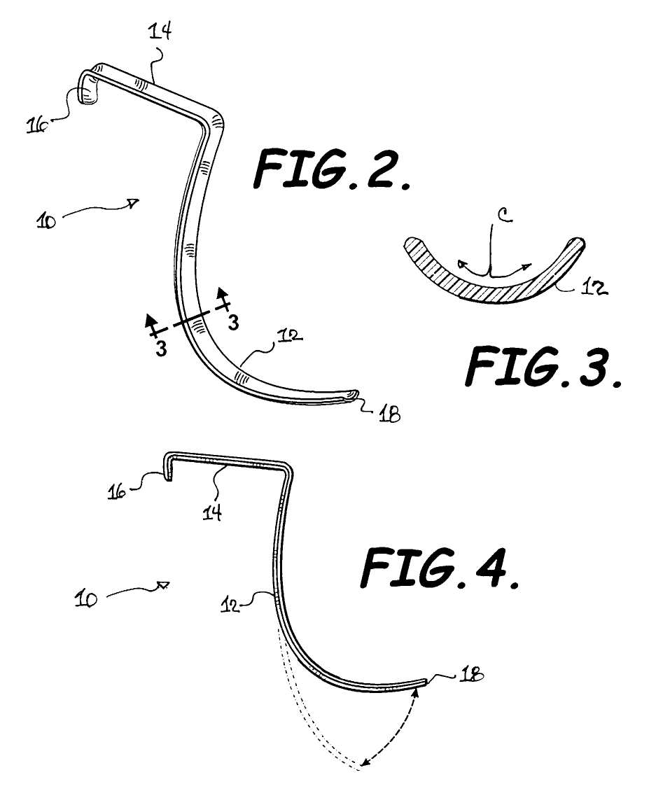

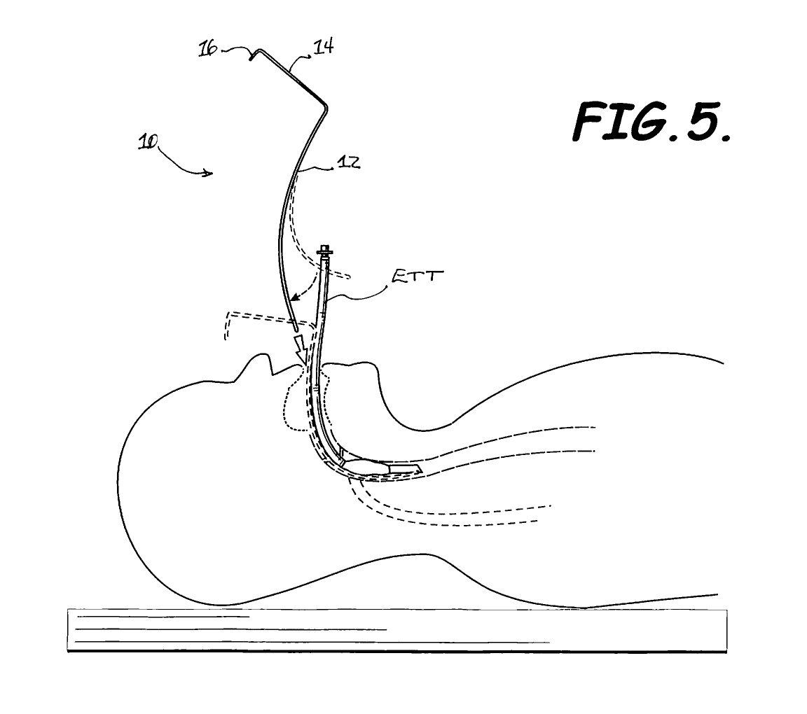

In a preferred embodiment shown in the figures, the present endotracheal tube exchanger (ETX) 10 comprises a tube slide portion 12 having an elongated lengthwise dimension having a first end and a second end, a relatively narrow width dimension having a curved cross section, and a generally arcuate shape having inner and outer peripheries. The curved cross section forms a concavity C along the inner periphery, as shown in FIG. 3. A handle portion 14 extends outwardly from the outer periphery of the first end of the tube slide portion 12, as seen in FIGS. 2 and 4. The tube slide portion 12 best comprises a relatively flexible plastic material which is resilient and provides sufficient flexibility to the device to allow the tube slide portion to adopt a shape complementary to that of an endotracheal tube ETT inserted in a patient, as depicted in FIGS. 1, 4 and 5. For example, as illustrated in FIGS. 4 and 5, the tube slide portion 12 is flexible enough to follow the curvature of the ETT as the exchanger is advanced into the patient's airway. Those skilled in the art will appreciate that a material such as Silastic® silicone rubber may be one of many materials which may be used in fabricating at least the tube slide portion of the presently described device. By contrast, the handle portion should be sufficiently rigid to facilitate handling by a user and may even be internally reinforced with a substantially rigid material. More preferably, the handle portion also includes a stop 16 to aid in preventing a user's hand from slipping off the handle portion. For example, the stop may be a terminal bend or hook at an end opposite from the tube slide portion, as seen in FIGS. 1-5. Additionally, a terminal end 18 of the tube slide portion 12 preferably is made generally rounded to facilitate insertion and advancement in the patient, while minimizing possible tissue trauma.

In use in a patient intubated with an endotracheal tube, the invention includes a method of replacing the existing endotracheal tube (ETT) with a new ETT. The method of the invention is illustrated in FIG. 5 and includes first advancing the ETX 10 into the intubated patient's airway by coupling the ETX to an outside surface of the ETT so that the concavity C of the ETX slides along an outer periphery of the existing ETT guiding the ETX to follow the ETT into the patient's airway. Then, withdrawing the existing ETT from the patient's airway by sliding outwardly along the ETX concavity while leaving the ETX in the patient's airway. This is followed by intubating the patient with the new ETT by positioning the new ETT on the concavity C of the ETX 10 and advancing the new ETT so that the concavity guides the ETT into the patient's airway. Finally, the method ends by withdrawing the ETX 10 from the patient while holding the new ETT in place in the patient's airway.

The invention may also be slightly differently described as including an endotracheal tube exchanger (ETX) 10 which has an arcuate slide 12 having first and second ends defining therebetween a curve having an inner periphery and an outer periphery, the inner periphery of the arcuate slide being generally concave C. A handle 14 extends away from the arcuate slide 12 at the first end, the handle providing a sufficient extent to receive thereon at least part of an operator's hand, Additional features of this embodiment are as described above for the first preferred embodiment.

Another method of replacing an existing endotracheal tube (ETT) in an intubated patient with a new ETT, comprises first positioning the endotracheal tube exchanger (ETX) 10 so that the concavity C of the ETX nests with a periphery of the existing ETT. Then, advancing the ETX 10 into the patient's airway so that the concavity C of the ETX slides along an outside surface of the ETT until the ETX extends into the patient's airway approximately equally with the existing ETT. The ETX 10 is held in place while pulling the existing ETT so that it slides along the concavity C of the ETX until fully withdrawn from the patient. Once the old ETT is removed, the method calls for positioning the new ETT so that it rests in the concavity C of the ETX 10 and advancing the new ETT into the patient's airway a sufficient distance by sliding the new ETT along the concavity C of the ETX 10. Finally, the method calls for holding the ETT in place while pulling the ETX 10 so that it slides along the ETT until the ETX is fully withdrawn from the patient.

Those skilled in the art will recognize that the presently described ETX 10 may include markings along its length to indicate how much of the device is inserted in the patient's airway. By using those markings, the health care provider can estimate how deeply to advance the ETX 10 into the patient's airway. Additionally, as is typical in practice, the ETX 10 may be coated with any suitable biocompatible lubricant for insertion in a patient and the device may also be manufactured of a slick material that will facilitate insertion.

Accordingly, in the drawings and specification, there have been disclosed typical preferred embodiments of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That which is claimed:

1. An endotracheal tube exchanger (ETX) comprising:
a tube slide portion having an elongated lengthwise dimension having a first end and a second end, a relatively narrow width dimension having a curved cross section, and a generally arcuate shape having inner and outer peripheries wherein the curved cross section forms a concavity along the inner periphery; and
a handle portion extending outwardly from the outer periphery of the first end of said tube slide portion.

2. The endotracheal tube exchanger of claim 1, wherein said tube slide portion comprises a relatively flexible plastic material.

3. The endotracheal tube exchanger of claim 1, wherein said tube slide portion comprises a resilient plastic material providing sufficient flexibility to allow said tube slide portion to adopt a shape complementary to that of an endotracheal tube inserted in a patient.

4. The endotracheal tube exchanger of claim 1, wherein said handle portion is sufficiently rigid to facilitate handling by a user.

5. The endotracheal tube exchanger of claim 1, wherein said handle portion further comprises a stop preventing a user's hand from slipping off said handle portion.

6. The endotracheal tube exchanger of claim 1, wherein said handle portion includes a terminal bend at an end opposite from said tube slide portion.

7. The endotracheal tube exchanger of claim 1, wherein a terminal end of said tube slide portion is generally rounded to facilitate insertion in a patient.

8. The endotracheal tube exchanger of claim 1, wherein said tube slide portion includes markings indicative of distance advanced into the patient's airway.

9. In a patient intubated with an endotracheal tube, a method of replacing the existing endotracheal tube (ETT) with a new ETT, the method comprising:
advancing the ETX of claim 1 into the intubated patient's airway by coupling the ETX to an outside surface of the ETT so that the concavity of the ETX slides along an outer periphery of the existing ETT guiding the ETX to follow the ETT into the patient's airway;
withdrawing the existing ETT from the patient's airway by sliding outwardly along the ETX concavity while leaving the ETX in the patient's airway;
intubating the patient with the new ETT by positioning the new ETT on the concavity of the ETX and advancing the new ETT so that the concavity guides the ETT into the patient's airway; and
withdrawing the ETX from the patient while leaving the new ETT inserted in the patient's airway.

10. An endotracheal tube exchanger (ETX) comprising:
an arcuate slide having first and second ends defining therebetween a curve having an inner periphery and an outer periphery, the inner periphery of said arcuate slide being generally concave; and
a handle extending away from said arcuate slide at the first end, said handle providing a sufficient extent to receive thereon at least part of an operator's hand.

11. The endotracheal tube exchanger of claim 10, wherein said arcuate slide comprises a relatively flexible plastic material.

12. The endotracheal tube exchanger of claim 10, wherein said arcuate slide comprises a resilient plastic material providing sufficient flexibility to allow said arcuate slide to adopt a shape complementary to that of an endotracheal tube inserted in a patient.

13. The endotracheal tube exchanger of claim 10, wherein said handle is sufficiently rigid to facilitate handling by a user.

14. The endotracheal tube exchanger of claim 10, wherein said handle further comprises a stop preventing a user's hand from slipping off said handle.

15. The endotracheal tube exchanger of claim 10, wherein said handle includes a terminal bend at an end opposite from said arcuate slide.

16. The endotracheal tube exchanger of claim 10, wherein a terminal end of said arcuate slide is generally rounded to facilitate insertion in a patient.

17. The endotracheal tube exchanger of claim 10, wherein said tube slide portion includes markings indicative of distance advanced into the patient's airway.

18. A method of replacing an existing endotracheal tube (ETT) in an intubated patient with a new ETT, the method comprising:
positioning the endotracheal tube exchanger (ETX) of claim 10 so that the concavity of the ETX nests with a periphery of the existing ETT;
advancing the ETX into the patient's airway so that the concavity of the ETX slides along an outside surface of the ETT until the ETX extends into the patient's airway approximately equally with the existing ETT;
holding the ETX in place while pulling the existing ETT so that it slides along the concavity of the ETX until fully withdrawn from the patient;
positioning the new ETT so that it rests in the concavity of the ETX;
advancing the new ETT into the patient's airway a sufficient distance by sliding the new ETT along the concavity of the ETX; and
holding the ETT in place while pulling the ETX so that it slides along the ETT until fully withdrawn from the patient.
